SEQUENCE LISTING (6 sequences)

SEQ ID NO: 1 - DNA, 39 bp, Artificial Sequence
  Description of Artificial Sequence Oligonucleotide
tgagggttaattgcgcgcttggcgtaatcatggtcatag

SEQ ID NO: 2 - DNA, 41 bp, Artificial Sequence
  Description of Artificial Sequence Oligonucleotide
ttgtaaaacgacggccagtgagcgcgcgtaatacgactcac

SEQ ID NO: 3 - DNA, 40 bp, Artificial Sequence
  Description of Artificial Sequence Oligonucleotide
ggccagtggttgtggaacggtatacttcatgtaagcgttc

SEQ ID NO: 4 - DNA, 41 bp, Artificial Sequence
  Description of Artificial Sequence Oligonucleotide
gagttccggagctggccacaaaccagagaatctctatcgag

SEQ ID NO: 5 - DNA, 43 bp, Artificial Sequence
  Description of Artificial Sequence Oligonucleotide
gatccaatacgactcactatagcggaagactctcctccggagg

SEQ ID NO: 6 - DNA, 43 bp, Artificial Sequence
  Description of Artificial Sequence Oligonucleotide
gatccctccggaggagagtcttccgctatagtgagtcgtattg

(19) United States
(12) Patent Application Publication
(10) Pub. No.: US 2009/0215029 A1
(43) Pub. Date: Aug. 27, 2009

(54) METHODS OF ISOLATING AND PURIFYING NUCLEIC ACID-BINDING BIOMOLECULES AND COMPOSITIONS INCLUDING SAME

(75) Inventors: Matthew M. Lawler, Jamaica Plain, MA (US); Timothy J. Haggerty, Concord, MA (US); Ian S. Dunn, New South Wales (AU)

Correspondence Address: PILLSBURY WINTHROP SHAW PITTMAN LLP, ATTENTION: DOCKETING DEPARTMENT, P.O BOX 10500, McLean, VA, 22102, US

(73) Assignee: CytoCure, Inc., Beverly, MA (US)

(21) Appl. No.: 12/015,230

(22) Filed: Jan. 16, 2008

Related U.S. Application Data
(60) Provisional application No. 60/885,160, filed on Jan. 16, 2007.

(51) Int. Cl. C12Q 1/70; C12Q 1/68; C12N 13/00

(52) U.S. Cl. 435/5; 435/6; 435/173.9; 435/239

(57) ABSTRACT

The invention provides methods for isolating or purifying a biomolecule directly or indirectly bound to a region of interest of a nucleic acid in a cell, and methods for isolating or purifying a biomolecule that directly or indirectly binds to a region of interest of a nucleic acid in a cell. The invention further provides substantially cell free, isolated and purified biomolecule(s) that are fixed or cross-linked to a region of interest of a nucleic acid, which optionally reflects the interaction of the biomolecule(s) with the nucleic acid in the cell (e.g., in native chromatin) when fixed or cross-linked to the region of interest. Substantially cell free, isolated and purified biomolecules that are fixed or cross-linked to a region of interest of a nucleic acid can remain fixed or cross-linked to the region of interest of the nucleic acid when heated or treated with denaturing compounds or agents.

### RELATED APPLICATIONS

This application claims the benefit of priority to application Ser. No. 60/885,160, filed Jan. 16, 2007, which is expressly incorporated herein by reference in its entirety.

### FIELD OF THE INVENTION

The invention relates to isolation and purification of biomolecules and molecular complexes that interact with nucleic acid sequences, including cellular and viral DNA and RNA, and methods of use thereof. The invention allows for identification and analysis of the biomolecules that bind to a particular nucleic acid sequence, as well as for identifying and screening compounds that modulate the binding of biomolecules to a particular nucleic acid sequence.

### INTRODUCTION

The current state of the art in chromatin analysis relies primarily on immunoprecipitation techniques for isolation of genomic loci (Das et al., BioTechniques. 2004, 37(6):961-9; Im et al., Methods Mol Biol. 2004; 284:129-46; Kuo and Allis, Methods. 1999; 19(3):425-33; Kuras L, Methods Mol Biol. 2004; 284:147-62; Weinmann and Farnham, Methods. 2002, 26(1):37-47). Chromatin immunoprecipitation utilizes an isolation protocol based on the bound molecule. Consequently, the method can only confirm or deny the presence of that particular molecule and is not capable of identifying additional bound factors. Furthermore, since a nucleic acid binding molecule may bind multiple target regions in a genome, multiple genomic loci are recovered. Even if additional bound factors could be identified from the isolate, they could not be ascribed to bind to a particular nucleic acid region of interest as they may be bound to any of the loci bound by the particular molecule.

Furthermore, chromatin immunoprecipitation requires the known or inferred presence of a particular bound molecule at a chromatin site of interest. An antibody of sufficient binding affinity for immunoprecipitation is also required. If an active factor is not known or suspected, or an antibody is not available, investigation cannot proceed. No de novo discovery of bound molecules is possible.

Alternative approaches have been developed to circumvent the limitations of chromatin immunoprecipitation (Wells and Farnham, Methods., 26:48 (2002); Liu et al., Cancer Res. 61:5402 (2001); Golden et al., Protein Sci. 8:2806 (1999); Forde and McCutchen-Maloney, Mass Spectrom Rev. November-December; 21:419 (2002); Stead et al., Mol Cell Proteomics. 5:1697 (2006); Gadgil et al., J Biochem Biophys Methods. 49:607 (2001)). While these approaches potentially allow analysis of a full complement of DNA-binding molecules, they suffer from several drawbacks such that binding patterns may not reflect events that occur in vivo with a consequent loss of biological relevance.

### SUMMARY

The invention provides methods for isolating and purifying, and optionally characterizing or analyzing biomolecules that bind directly or indirectly to nucleic acids in a cell. The invention provides a way to isolate a single nucleic acid locus from a cell, plurality of cells or a sample containing cells, and analyze and identify all of the molecular factors bound specifically to that locus. For example, a method of the invention can faithfully capture nucleic acid-protein binding as it occurs in vivo. The invention provides methods to isolate one or more biomolecules bound in vivo to any desired or target nucleic acid sequence or region, without a priori knowledge or inference of specific binding factor(s) that are or may be present.

The invention also provides substantially cell free compositions that include one or more biomolecules fixed or cross-linked to a region of interest of a nucleic acid, in which one or more of the biomolecules remain fixed or cross-linked to the region of interest of the nucleic acid when treated with a denaturant or subject to heat. The invention further provides compositions that include isolated or purified biomolecule(s) fixed or cross-linked to a region of interest of a nucleic acid, in which one or more of the biomolecules remain fixed or cross-linked to the region of interest of the nucleic acid when treated with a denaturant or subject to heat. The invention moreover provides isolated and purified biomolecule(s) fixed or cross-linked to a region of interest of a nucleic acid that reflects the interaction of the biomolecule with the nucleic acid in a cell in a native state, such as in chromatin, when the biomolecule(s) is fixed or cross-linked to the region of interest of the nucleic acid.

The invention methods and isolated and purified biomolecules bound to one or more nucleic acid regions of interest in a cell or biological sample reflect molecular interactions in the native state, such as in chromatin, which are preserved or maintained. Nucleic acid interactions with one or more biomolecules are preserved or maintained by covalent fixation or cross-linking of biomolecules to the nucleic acid before lysis or disruption of the cell. Following fixation, complexes of biomolecule bound to nucleic acid are harvested, and the nucleic acid is fragmented. A nucleic acid sequence complementary to a region of interest in the complex is used as a “capture” oligonucleotide to isolate desired nucleic acid fragments among the sample. Recovery of bound biomolecule(s) from isolated or purified nucleic acid/binding biomolecule complex allows analysis of all molecular factors associated with a nucleic acid region of interest in a cell. Bound biomolecules can then be identified by methods such as mass spectroscopy. Instead of examining the action of a single binding factor at many different nucleic acid loci in a genome, the repertoire of nucleic acid binding biomolecules at a specific nucleic acid locus can be examined.

The invention further provides methods for determining or analyzing the effect of a test compound (e.g., a drug) or treatment on the binding of a biomolecule directly or indirectly to a region of interest of a nucleic acid in a cell. For example, binding of a biomolecule to a region of interest of a nucleic acid can be ascertained in the absence and presence of a test compound or test treatment, and the effect of the test compound or treatment on binding of the biomolecule to the nucleic acid determined. If the test compound or treatment decreases or increases binding of the biomolecule to the nucleic acid, the test compound or treatment is identified as a compound or treatment that modulates binding of the biomolecule to the nucleic acid. More particularly, a method for identifying or screening a compound or treatment that modulates binding of a biomolecule directly or indirectly to a region of interest of a nucleic acid in a cell includes providing a cell or plurality of cells or a sample; contacting the cell, cells or sample with a test compound or treatment; contacting or exposing the cell, cells or sample to a fixing or cross-linking agent or treatment under conditions in which direct or indirect binding of one or more biomolecules to the nucleic acid is preserved or maintained, thereby producing fixed or cross-linked nucleic acid; optionally isolating the fixed or cross-linked nucleic acid; fragmenting the fixed or cross-linked nucleic acid, thereby producing fragmented fixed or cross-linked nucleic acid; optionally denaturing the fragmented fixed or cross-linked nucleic acid; hybridizing a capture oligonucleotide to the fragmented fixed or cross-linked nucleic acid to form a hybridized target, wherein the capture oligonucleotide is complementary to all or a part of the region of interest of the nucleic acid; isolating or purifying the hybridized target; identifying or characterizing one or more biomolecules directly or indirectly bound to the region of interest of the nucleic acid; and comparing binding of the one or more biomolecules to binding in the absence of the test compound or treatment-altered (e.g., decreased or increased) binding in the presence of the test compound or treatment identifies the compound or treatment as a compound or treatment that modulates binding of the biomolecule to the region of interest of the nucleic acid in the cell.

In such methods for determining or analyzing the effect of a test compound (e.g., a drug) or treatment on the binding of a biomolecule directly or indirectly to a region of interest of a nucleic acid in a cell, the identity of the biomolecule that binds to the nucleic acid with the region of interest may but need not be known prior to screening. Thus, for example, the effect of a test compound or treatment on binding of a biomolecule to a nucleic acid, wherein said biomolecule is already known to bind to the nucleic acid, can be determined in a cell, in which the nucleic acid is in a native state, e.g., chromatin.

The invention moreover provides fixative agents. In particular embodiments, a fixative agent includes first and second functional groups, wherein the first functional group includes an azide, and the second functional group includes an N-hydroxysuccinimide (NHS) ester. In further particular embodiments, a fixative agent includes first and second functional groups, wherein the first functional group comprises nitrogen mustard, an azide or cis-platinum, and the second functional group comprises an N-hydroxysuccinimide (NHS) ester. In additional particular embodiments, a fixative agent includes first and second functional groups, wherein the first functional group comprises a platinum derivative, and the second functional group comprises an N-hydroxysuccinimide (NHS) ester.

### DESCRIPTION OF DRAWINGS

FIG. 1 shows an image a silver-stained polyacrylamide gel electrophoresis of material recovered from E. coli cell lysates using the method described. Lane A, 25 μL of negative control (fixed lysate hybridized to scrambled dummy probes) sample. Lane B, 25 μL of experimental sample (fixed lysate hybridized to lac operon-specific probes). The arrow shows the location of E. coli lac repressor protein (39 kDa).

FIG. 2 shows a schematic diagram of an exemplary invention method.

FIG. 3 shows nucleic acid fluorescent staining of free nucleic acid and covalent complexes of nucleic acid and bound biomolecules after cross-linking and heat denaturation. Arrows labeled A indicate covalently linked complexes, while the arrow labeled B indicates more rapidly migrating free nucleic acid. Lane 1: negative control (no cross-linking agent). Lane 2: treated with Novel Cross-linking Agent 1. Lane 3: treated with Novel Cross-linking Agent 2. Lane 4: treated with Novel Cross-linking Agent 3. Each lane is from a different portion of the same gel.

### DETAILED DESCRIPTION

The invention provides methods for isolating or purifying a biomolecule directly or indirectly bound to a region of interest of a nucleic acid in a cell. In one embodiment, a method includes providing a cell or plurality of cells (e.g., a sample of cells or tissue); contacting or exposing the cell or cells to a fixing or cross-linking agent or treatment under conditions in which direct or indirect binding of one or more biomolecules to the nucleic acid is preserved or maintained, thereby producing fixed or cross-linked nucleic acid; optionally isolating the fixed or cross-linked nucleic acid; fragmenting the fixed or cross-linked nucleic acid, thereby producing fragmented fixed or cross-linked nucleic acid; optionally denaturing the fragmented fixed or cross-linked nucleic acid; hybridizing a capture oligonucleotide to the fragmented fixed or cross-linked nucleic acid to form a hybridized target, wherein the capture oligonucleotide is complementary to all or a part of the region of interest of the nucleic acid; and isolating or purifying the hybridized target, thereby isolating or purifying a biomolecule directly or indirectly bound to a region of interest of the nucleic acid in the cell.

The invention also provides methods for isolating or purifying a biomolecule that directly or indirectly binds to a region of interest of a nucleic acid in a cell. In one embodiment, a method includes providing a cell or plurality of cells (e.g., a sample of cells or tissue); contacting or exposing the cell or cells to a fixing or cross-linking agent or treatment under conditions in which direct or indirect binding of one or more biomolecules to the nucleic acid is preserved or maintained, thereby producing fixed or cross-linked nucleic acid; optionally isolating the fixed or cross-linked nucleic acid; fragmenting the fixed or cross-linked nucleic acid, thereby producing fragmented fixed or cross-linked nucleic acid; optionally denaturing the fragmented fixed or cross-linked nucleic acid; hybridizing a capture oligonucleotide to the fragmented fixed or cross-linked nucleic acid to form a hybridized target, wherein the capture oligonucleotide is complementary to all or a part of the region of interest of the nucleic acid; isolating or purifying the hybridized target; and removing one or more biomolecules directly or indirectly bound to the hybridized target, thereby isolating or purifying a biomolecule that directly or indirectly binds to the region of interest of the nucleic acid in the cell.

As used herein, the terms “nucleic acid,” “polynucleotide” and “oligonucleotide” and the like refer to at least two or more ribo- or deoxy-ribonucleic acid base pairs (nucleotides) that are linked through a phosphoester bond or equivalent. Shorter polynucleotides (e.g., capture oligonucleotides) are commonly referred to as “oligonucleotides” or “probes” of single- or double-stranded DNA or RNA. However, there is no upper limit to the length of such polynucleotides. Nucleic acids include polynucleotides and polynucleosides. Nucleic acids can be or form single, double or triplex, circular or linear molecules. Exemplary nucleic acids include but are not limited to: RNA (e.g., mRNA, rRNA, tRNA, hnRNA, etc.), DNA, cDNA, genomic nucleic acid (DNA), naturally occurring and non naturally occurring nucleic acid, e.g., recombinant or synthetic nucleic acid. Nucleic acids include eukaryotic (e.g., mammalian, such as primate or human), bacterial or viral DNA or RNA.

As used herein, the term “region” or “area” of interest refers to a part of a nucleic acid in which it is desired to obtain information regarding whether a biomolecule binds at or near the region or area of interest, analysis of one or more biomolecules that bind at or near the region or area of interest, or the identity of one or more biomolecules that bind at or near the region or area of interest. A region of interest is therefore used to denote a portion of a nucleic acid to be analyzed for biomolecule binding.

A region of interest of a nucleic acid is specified by a capture oligonucleotide due to specific hybridization of the capture oligonucleotide to the region or area of interest. A region of interest of a nucleic acid can therefore be targeted or selected based upon the sequence of the capture oligonucleotide.

Capture oligonucleotides can also be of various lengths. Capture oligonucleotides can be relatively short, for example, 100 to about 500 nucleotides, or shorter, for example, from about 12 to 25, 25 to 50, 50 to 100, 100 to 250, or about 250 to 500 nucleotides in length, or any numerical value or range or value within or encompassing such lengths. In particular embodiments, a capture oligonucleotide has a length from about 10-20, 20-30, 30-50, 50-100, 100-150, 150-200, 200-250, 250-300, 300-400, 400-500, 500-1000, 1000-2000, nucleotides, or any numerical value or range within or encompassing such lengths.

Multiple capture oligonucleotides can be employed in the methods of the invention. For example, two or more capture oligonucleotides that hybridize to the same or a different portion of a particular region of interest.

Capture oligonucleotides include L- or D-forms and mixtures thereof, and additionally may be modified to be resistant to degradation. Particular examples include 5′ and 3′ linkages resistant to endonucleases and exonucleases. Additional examples include peptide nucleic acids (PNAs) that bind RNA or DNA.

Nucleic acids and regions of interest can be of various sequence lengths. Nucleic acid lengths, and regions of interest, typically range from about 10 nucleotides to 100 Kb, or any numerical value or range within or encompassing such lengths, 20 nucleotides to 50 Kb, 50 nucleotides to 25 Kb, 100 nucleotides to 10 Kb, 500 nucleotides to 5 Kb, 1 to 5 Kb or less, such as 1000 to about 500 nucleotides. Nucleic acids and regions of interest can also be shorter, for example, 100 to about 500 nucleotides, or from about 12 to 25, 25 to 50, 50 to 100, 100 to 250, or about 250 to 500 nucleotides in length, or any numerical value or range or value within or encompassing such lengths. In particular embodiments, a nucleic acid sequence or a region of interest has a length from about 10-20, 20-30, 30-50, 50-100, 100-150, 150-200, 200-250, 250-300, 300-400, 400-500, 500-1000, 1000-2000, nucleotides, or any numerical value or range within or encompassing such lengths. A region of interest is typically within and therefore a subsequence of a nucleic acid.

Nucleic acids can be produced using various standard cloning and chemical synthesis techniques. Techniques include, but are not limited to nucleic acid amplification, e.g., polymerase chain reaction (PCR), with genomic DNA or cDNA targets using primers (e.g., a degenerate primer mixture) capable of annealing to antibody encoding sequence. Nucleic acids can also be produced by chemical synthesis (e.g., solid phase phosphoramidite synthesis) or transcription from a gene. The sequences produced can then be translated in vitro, or cloned into a plasmid and propagated and then expressed in a cell (e.g., a host cell such as yeast or bacteria, a eukaryote such as an animal or mammalian cell or in a plant).

Capture oligonucleotides can also include a heterologous domain. Thus, a heterologous domain can consist of any of a variety of different types of small or large functional moieties such as nucleic acid, peptide, carbohydrate, lipid or small organic or inorganic compounds, such as a drug, metals (gold, silver), etc.

Particular non-limiting examples of heterologous domains include, for example, tags and detectable labels. Specific examples of tags and detectable labels include enzymes (horseradish peroxidase, urease, catalase, alkaline phosphatase, beta-galactosidase, chloramphenicol transferase); enzyme substrates; affinity tags such as ligands (e.g., biotin, or a derivative or amino acid variant thereof) and receptors (e.g., avidin, neutravidin or streptavidin, or a derivative or amino acid variant thereof); radionuclides (e.g., C¹⁴, S³⁵, P³², P³³, H³, I¹²⁵, I¹³¹, gallium-67 and 68, scantium-47, indium-111, radium-223); T7-, His-, myc-, HA- and FLAG-tags; electron-dense reagents; energy transfer molecules; paramagnetic labels; electron spin-labels; fluorophores (fluorescein, rhodamine, phycoerythrin); chromophores; chemi-luminescent (imidazole, luciferase); and bio-luminescent agents.

Particular non-limiting examples of heterologous domains also include, for example, substrates. Specific examples of substrates include those appropriate for immobilization, such as sepharose and sephadex. Substrates further include a membrane, such as nylon, cellulose, nitrocellulose, or polyvinylidene difluoride (PVDF). Substrates may also include metallic surfaces, such as gold.

Substrates may also consist of a membrane, glass, plastic or other synthetic or natural polymer, metal or silica slide. Such substrates can have one or more (e.g., a plurality) of capture oligonucleotides positionally arrayed on the substrate surface. A single contiguous immobilized substrate (membrane, glass slide, plastic or other synthetic or natural polymer, metal, etc.), for example, a chip, may contain multiple capture oligonucleotides specific for multiple nucleic acid regions of interest. Each capture oligonucleotide can be localized at defined positions or regions (addresses) of the substrate, or synthesized on the surface at defined positions or regions (addresses) of the substrate in situ. Such substrates facilitate parallel analysis of bound biomolecules at multiple nucleic acid regions of interest from a single cell sample. Such substrates are also appropriate for high throughput screening and identifying bound biomolecules at multiple nucleic acid regions of interest.

Linker sequences may be inserted between the polynucleotide and heterologous domain so that the two entities maintain, at least in part, a distinct function or activity. Linker sequences may have one or more properties that include a flexible structure, an inability to form an ordered secondary structure or a hydrophobic or charged character which could promote or interact with either domain. Amino acids typically found in flexible protein regions include Gly, Asn and Ser. Other near neutral amino acids, such as Thr and Ala, may also be used in the linker sequence. The length of the linker sequence may vary (see, e.g., U.S. Pat. No. 6,087,329). Linkers further include chemical cross-linking and conjugating agents, such as sulfo-succinimidyl derivatives (sulfo-SMCC, sulfo-SMPB), disuccinimidyl suberate (DSS), disuccinimidyl glutarate (DSG) and disuccinimidyl tartrate (DST).

A nucleic acid is typically analyzed in a cell. In this way, the natural interaction that occurs between a biomolecule and a nucleic acid, for example, in chromatin, can be analyzed in vivo. Thus, nucleic acid typically is present in a living or non-living prokaryotic or eukaryotic cell or a virus therein. Nucleic acids can also be present in a plurality or collection of cells, such as a cell sample. Exemplary non-limiting cell samples include a tissue or organ sample, a biological fluid, a cell lysate, soil, water, air, or a synthetically produced biomolecule or mixture of biomolecules.

Nucleic acid analyzed in a cell can be an endogenous gene or a gene that has been transformed into a cell by genetic manipulation, for example. Intracellular nucleic acid can be a single or multi-copy gene (e.g., 2, 3, 4, 5 or more gene copies).

As used herein, the term “biomolecule” in the context of a molecule that binds to a nucleic acid refers to an entity that can or does directly or indirectly bind to a nucleic acid sequence. Biomolecules can bind directly to nucleic acid, for example, due to affinity for nucleic acid (e.g., hydrogen bonds, ionic bonds, etc.), or indirectly, for example, by binding to a second biomolecule that itself binds directly to a nucleic acid.

A biomolecule may but need not modulate activity, structure or function of a nucleic acid (e.g., increase or decrease transcription, replication, etc.) to which it binds. Biomolecules are not limited to naturally occurring biological molecules, such as a proteins, nucleic acids, carbohydrates, fats, lipids, and others normally found in cells, but also include synthetic and naturally occurring compounds (e.g., drugs) not normally present in cells that can bind to a nucleic acid in a cell. For example, contact of cells (e.g., a cell or tissue sample) with a drug can result in the drug becoming intracellular and binding to a nucleic acid (directly) in the cell, or binding to a biomolecule that in turn binds to nucleic acid (indirectly) in the cell. Such a biomolecule that binds to a nucleic acid in a cell can be detected in accordance with the invention.

A biomolecule may include or consist of a single molecular entity, or a monomer. A biomolecule can interact covalently or non-covalently with other biomolecules to form a complex in which one or more components of the complex mediates or participates in binding to the nucleic acid. Other biomolecules within the complex need not contact or bind to or have affinity for the nucleic acid, but can contact one or more biomolecules that contact or bind to or have affinity for the nucleic acid. A biomolecule can therefore include or consist of a plurality of individual molecular entities, such as a dimer, trimer, teramer, pentamer, etc., comprising monomers, or a complex of two, three, four, five, etc., or more different or distinct individual entities, which can be conveniently referred to herein as a molecular complex.

Exemplary nucleic acid binding biomolecules include polypeptides. The terms “polypeptide,” “protein” and “peptide” are used interchangeably herein to refer to two or more amino acids or “residues,” covalently linked by an amide bond or equivalent. Polypeptides can be various lengths and the amino acids may be linked by non-natural and non-amide chemical bonds including, for example, those formed with glutaraldehyde, N-hydroxysuccinimide esters, bifunctional maleimides, or N,N′-dicyclohexylcarbodiimide (DCC). Non-amide bonds include, for example, ketomethylene, aminomethylene, olefin, ether, thioether and the like (see, e.g., Spatola in Chemistry and Biochemistry of Amino Acids, Peptides and Proteins, Vol. 7, pp 267-357 (1983), “Peptide and Backbone Modifications,” Marcel Decker, NY).

Exemplary biomolecules include nucleic acids. Such nucleic acid biomolecules can be or form single, double or triplex, circular or linear biomolecules. Nucleic acid biomolecules include but are not limited to: RNA (e.g., mRNA, rRNA, tRNA, hnRNA, etc.), DNA, cDNA, genomic nucleic acid, naturally occurring and non naturally occurring nucleic acid, e.g., recombinant or synthetic nucleic acid.

Exemplary biomolecules further include small molecules such as hormones, cytokines, and drugs. A biomolecule can also be a non-proteinaceous molecule, such as a lipid, fat, carbohydrate or nucleic acid, or any combination thereof.

Exemplary classes of nucleic acid binding biomolecules include trans-acting and regulatory elements (e.g., transcription factors) that modulate (increase or decrease) transcription, replication factors, translation factors, restriction (exo- and endo-nucleases) and modifying factors (DNA and RNA polymerases, ligases, topoisomerases, telomerases) and other molecules that bind to a nucleic acid sequence. Exemplary nucleic acid binding biomolecules also include structural and assembly factors such as components of chromatin and chromosomes (e.g., histones such as H1, H2A, H2B, H3, H4, H5, scaffold proteins, centromeres and telomeres).

Biomolecules that bind to nucleic acid can be modified or derivatized. For example, biomolecules can be phosphorylated, methylated, acetylated, nitrated, or have of one or more carbohydrates (glycosylated), nucleotides, nucleic acids, cofactors (e.g., vitamins), fats or lipids attached thereto.

Biomolecules that directly or indirectly bind to nucleic acid can be fixed or cross-linked to nucleic acid using a variety of fixative or agents or crosslinkers. For example, an agent or treatment or combination of agents or treatments that preserves the interaction between nucleic acid and bound biomolecules, by establishing a covalent bond, can be utilized as a fixative or cross-linker.

Particular non-limiting examples include exposure to radiation, such as ultraviolet (UV) radiation, laser radiation or light radiation. Additional particular non-limiting examples include a fixing or cross-linking agent that has at least two functional groups, for example, a nucleic acid reactive or binding group and a protein reactive or binding group. A further particular non-limiting example of a fixing or cross-linking agent or treatment is a chemical cross-linker (e.g., a reversible cross-linker). Methods and compositions of the invention include treatment with two or more fixing agents, two or more cross linkers or two or more cross-linking agents.

Chemical groups and compounds that comprise a fixing or cross-linking agent or treatment include one or more of: an aldehyde-containing compound; an amine reactive group; a sulfhydryl reactive group; a hydroxyl reactive group; a carboxyl reactive compound; a carbohydrate reactive group; an alkylating agent; a nucleic acid base reactive group; a nucleic acid backbone alkylating agent; or a nucleic acid adduct-forming metallic compound. Specific examples of aldehyde-containing compounds include formaldehyde, glyoxal or glutaraldehyde. For example, in a particular aspect, fixation is performed with an aldehyde fixation followed by cross-link bond stabilization with a reducing agent, such as sodium cyanoborohydride or sodium borohydride. Specific examples of amine reactive groups include N-hydroxy succinimide, an imidoester, hydroxymethyl phosphine, a nitrogen mustard or derivative thereof, and a sulfur mustard or derivative thereof. Specific examples of sulfhydryl reactive compounds include maleimide, vinylsulfone and pyridyl disulfide. Specific examples of hydroxyl reactive groups include isocyanate. Specific examples of a carboxyl reactive compound is carbodiimide. Specific examples of a carbohydrate reactive group is hydrazine. Specific examples of a nucleic acid backbone alkylating agent includes an N-nitrosourea, or a sulfonyl chloride. Specific examples of nucleic acid adduct-forming metallic compounds include cis-platinum, trans-platinum, or a platinum derivative, potassium chromate and hexavalent chromium.

Still further particular non-limiting examples of a fixing or cross-linking agent or treatment is an azide, such as a photoactive azide, an epoxide (e.g., butadiene diepoxide), psoralen, trioxsalen or vinyl sulfone; an organometallic group or compound, such as cis-platinum or a derivative thereof, or an organometallic copper compound; and a reactive halogen, such as N-bromo-succinimide. Yet further particular non-limiting examples of a fixing or cross-linking agent or treatment is bisulfite, sodium bisulfite, permanganate, hydrazine, dimethyl sulfate, or a modified dimethyl sulfite.

Chemical groups and compounds that comprise a fixing or cross-linking agent or treatment also include a modified nucleotide or amino acid that is incorporated into cellular nucleic acids or proteins. Particular non-limiting examples include 5-bromo-deoxyuridine, photoleucine and photomethionine.

The invention further provides fixing and cross-linking agents and treatments. In particular embodiments, a fixative agent or a cross-linking agent is or includes a compound having nitrogen mustard and N-hydroxysuccinimide (NHS) ester, azide and N-hydroxysuccinimide (NHS) ester, or cis-platinum and N-hydroxysuccinimide (NHS) ester. In additional embodiments, the fixing or cross-linking treatment is or includes treatment with ethidium bromide monoazide and ethylene glycol bis-[succinimidyl succinate] (EGS), cis-platin and ethylene glycol bis-[succinimidyl succinate] (EGS), nitrogen mustard and ethylene glycol bis-[succinimidyl succinate] (EGS) or 4-aminomethyl trioxsalen and ethylene glycol bis-[succinimidyl succinate] (EGS). Such fixing and cross-linking agents and treatments can be used in accordance with the invention methods and compositions.

Fixing and cross-linking agents and treatments can be a group or include a compound with affinity for a nucleic acid. In particular embodiments, a group with high chemical affinity for a nucleic acid is or includes an intercalator (e.g., ethidium bromide, propidium iodide, angelicin, aziridine, quinacrine, or an anthracycline), a nucleic acid duplex groove-binding entity (e.g., Hoechst 33258, Hoechst 33342, 4′,6-diamidino-2-phenylindole, or a lexitropsin), or a phosphate backbone interacting entity (e.g., phosphate backbone interacting entity comprises spermine, polyethylene imine, or quaternary ammonium).

Fixing and cross-linking agents and treatments can be labeled or tagged. Examples of labels and tags are set forth herein and known to one skilled in the art.

Following fixing or cross-linking, the nucleic acid is optionally isolated. Essentially, the nucleic acid is isolated or collected from the cell by an appropriate method. For example, an antibody can be used to bind to a biomolecule fixed or cross-linked to the nucleic acid with and isolated. Cells can be lysed prior to or following isolation or collection. In a particular embodiment, for example, cells are lysed after the fixed or cross-linked nucleic acid is isolated or collected. Cells can be lysed by a variety of methods including, but not limited to, physical means, mechanical means, chemical or enzymatic means, freeze-thaw, mechanical homogenation, sonication, French press disruption, hypotonic or alkaline lysis, detergent lysis, and chaotropic salt lysis.

Following fixing or cross-linking, or isolating the fixed or cross-linked nucleic acid, the nucleic acid is fragmented. Nucleic acid can be fragmented by a variety of methods including, but not limited to, physical means (shearing), mechanical means (e.g., sonication or syringe passage), chemical cleavage or enzymatic cleavage (e.g., with an exonuclease or endocnuclease, or a nucleic acid sequence specific or non-specific restriction enzyme or nuclease).

Following fragmentation of the fixed or cross-linked nucleic acid, the fragmented nucleic acid can be subject to denaturation prior to hybridization with a capture oligonucleotide. Denaturation of nucleic acid can be performed various ways including heat or chemical means.

The term “hybridize” and grammatical variations thereof refer to the binding between nucleic acid sequences. Hybridizing sequences will generally be more than about 50% complementary (e.g., 50%, 60%, 70%, 80%, 90%, 95%, or more precise base pairing) to a reference nucleic acid sequence or a sequence homologous or identical to a reference sequence. Capture oligonucleotides can but need not be 100% complementary to all or a portion of a region of interest or a sequence to which it hybridizes. Capture oligonucleotides with a sequence less than 100% complementarity to a region of interest can exhibit sufficient complementarity so as to be able to hybridize to all or a portion of a region of interest. Such sequences less than 100% complementary, which can be referred to as “partially” complementary, include 95%, 90%, 80%, 70% or less complementary, but are typically 80-100% complementary to the region of interest. In general, as the sequence length increases, the percentage complementarity required for hybridization decreases. Hybridizing sequences, such as a capture oligonucleotide that is 100% or fully complementary to a reference sequence, such as a region of interest exhibits 100% base pairing with no mismatches.

The hybridization region between hybridizing sequences typically is at least about 10-15 nucleotides, 15-20 nucleotides, 20-30 nucleotides, 30-50 nucleotides, 50-100 nucleotides, 100 to 200 nucleotides, 200 to 500 nucleotides or more, or any numerical value or range within or encompassing such lengths. The hybridization sequences need not span the entire length of the capture oligonucleotide or region of interest and can therefore be a portion of each, one or more nucleotides (2, 3, 4, 5, 5-10, 10-20, 20-30, etc.) less than the full length sequence.

Following hybridization, the hybridized target is separated from non-hybridized fragmented fixed or cross-linked nucleic acid method. Appropriate methods for such separation include physically separating the hybridized target from non-hybridized fragmented fixed or cross-linked nucleic acid.

Hybridization can be performed multiple (e.g., two, three, four, five or more) times. This removes non-hybridizing sequences or other contaminants, which may improve purity. The multiple hybridizations can be performed with the same or a different capture oligonucleotide.

Hybridization can be performed with multiple capture oligonucleotides. For example, a first hybridization can be performed with a first capture oligonucleotide, and a second hybridization can be performed with a second capture oligonucleotide. Such multiple hybridizations can include a washing step to remove any undesirable (e.g., non-hybridizing sequences) components.

Methods of the invention can include a hybridization-promoting enzyme in order to increase or stimulate hybridization between a capture oligonucleotide and a nucleic acid. In a particular aspect, a hybridization-promoting enzyme is recA.

In particular embodiments, a biomolecule(s) bound to the nucleic acid can be removed from the nucleic acid to which it has been fixed or cross-linked. Non-limiting techniques for biomolecule removal include thermal, chemical, enzymatic (e.g., a protease such as trypsin, lysozyme, or V8 protease) or mechanical disruption of the biomolecule bound (fixed or cross-linked) to the hybridized target, or any combination thereof.

If desired, the biomolecule(s) bound to the nucleic acid, or a biomolecule removed from the nucleic acid to which it has been fixed or cross-linked can be isolated, purified or characterized (e.g., sequenced). In a particular embodiment, a method includes characterizing or identifying the isolated or purified biomolecule bound to the region of interest of the nucleic acid. In another particular embodiment, a method includes characterizing or identifying one or more of the removed biomolecules. Non-limiting techniques appropriate for such analysis include gel electrophoresis, western blotting, mass spectrometry, matrix-assisted Laser Desorption Ionization (MALDI), chromatography, nuclear magnetic resonance and crystallography.

Methods of determining binding of a biomolecule to nucleic acid include size fractionation (unbound nucleic acid will have a smaller size than bound nucleic acid). A nucleic acid that is bound to a biomolecule will be distinguished from unbound nucleic acid by virtue of size, shape, charge or density. For example, a nucleic acid bound to biomolecules will pass through a chromatography column at a different rate than unbound nucleic acids. Additionally, depending on the nature of the biomolecule, a nucleic acid bound to a biomolecule can have a greater or lesser density than an unbound nucleic acid, and can be separated from unbound nucleic acids by density centrifugation. Furthermore, bound and unbound nucleic acids will have different electrophoretic mobilities, and can be separated by methods such as electrophoretic mobility shift (gel shift) assays (EMSA).

Methods of determining binding of a biomolecule to the cell also include nuclease resistance. As a non-limiting example a nucleic acid can contain a sequence that is cleaved by a restriction enzyme. If the region of the nucleic acid with the sequence binds to a biomolecule, the restriction enzyme will be unable to cleave the site. In contrast, if the region of the nucleic acid with the sequence does not bind to a biomolecule, the restriction enzyme will be able to cleave the site. Thus, nucleic acid that is not bound to a biomolecule is cleaved, whereas nucleic acid that is not bound to a biomolecule is not cleaved. A polymerase chain reaction (PCR) reaction can be performed to amplify only those nucleic acids that were not cleaved and therefore not bound by the biomolecule.

Analysis following isolating or purifying the hybridized target includes, among others, characterizing or identifying the nucleic acid sequence or residues thereof to which one or more of the biomolecules bind (e.g., by identifying residues to which the cross linker is attached), characterizing or identifying one or more of the bound biomolecules (e.g., by mass spectrometry).

In accordance with the invention, there are provided substantially cell free compositions that include a biomolecule fixed or cross-linked to a region of interest of a nucleic acid. In one embodiment, a composition is substantially cell free and includes a biomolecule fixed or cross-linked to a region of interest of a nucleic acid, wherein at least a proportion of the biomolecule (e.g., 50%, 60%, 70%, 80%, 90% or more) remains fixed or cross-linked to the region of interest of the nucleic acid when heated to a temperature of 67 degrees Celsius for 4 hours. In another embodiment, a composition is substantially cell free and includes a biomolecule fixed or cross-linked to a region of interest of a nucleic acid, wherein at least a proportion of the biomolecule (e.g., 50%, 60%, 70%, 80%, 90% or more) remains fixed or cross-linked to the region of interest of the nucleic acid when heated to a temperature of 100 degrees Celsius for 15 minutes.

The term “cell free” as used herein means that the composition does not contain more than 25% living cells in the population. Thus, cell free can include 75% dead cells or cellular debris. Typically cell free does not have more that 20%, 15%, 10% or 5% living cells.

In accordance with the invention, there are also provided isolated and purified compositions that include a biomolecule fixed or cross-linked to a region of interest of a nucleic acid. In one embodiment, an isolated or purified composition includes a biomolecule fixed or cross-linked to a region of interest of a nucleic acid, wherein at least a proportion of the biomolecule (e.g., 50%, 60%, 70%, 80%, 90% or more) remains fixed or cross-linked to the region of interest of the nucleic acid when heated to a temperature of 67 degrees Celsius for 4 hours. In another embodiment, an isolated or purified composition includes a biomolecule fixed or cross-linked to a region of interest of a nucleic acid, wherein at least a proportion of the biomolecule (e.g., 50%, 60%, 70%, 80%, 90% or more) remains fixed or cross-linked to the region of interest of the nucleic acid when heated to a temperature of 100 degrees Celsius for 15 minutes.

Compositions that include a biomolecule fixed or cross-linked to a region of interest of a nucleic acid include those that reflect the interaction of the biomolecule with the nucleic acid in a cell when fixed or cross-linked to the region of interest. For example, a composition that includes a biomolecule fixed or cross-linked to a region of interest of a nucleic acid include those that reflect the interaction of the biomolecule with the nucleic acid in native chromatin when fixed or cross-linked to the region of interest.

Such compositions can be obtained with any method of the invention. Accordingly, isolated and purified biomolecule(s) directly or indirectly bound to a region of interest of a nucleic acid produced by the methods of the invention are provided.

The term “isolated,” when used as a modifier of a composition means that the composition is made by the hand of man or is separated from one or more other components in their naturally occurring in vivo (e.g., cellular) environment. Generally, compositions so separated are substantially free of one or more materials with which they normally associate with in nature, for example, one or more cells, protein, nucleic acid, lipid, fat, carbohydrate, cell membrane. Thus, an isolated composition is separated from other biological components of a cell or organism in which the composition naturally occurs, or from the artificial medium in which it is produced (e.g., synthetically or through cell culture). For example, an isolated biomolecule that binds to or is bound to a nucleic acid region of interest is substantially separated from other nucleic acid and polypeptides and does not include a library of polynucleotides or polypeptides present among millions of polypeptide or nucleic acid sequences, such as a polypeptide, genomic or cDNA library, for example. The term “isolated” does not exclude alternative physical forms of the composition, for example, an isolated biomolecule could include protein multimers, oligomers, post-translational modifications (e.g., glycosylation, phosphorylation, acetylation, methylation) or derivatized forms.

The term “purified,” when used as a modifier of a composition refers to a composition free of most or all of the materials with which it typically associates with in nature (e.g., cellular environment). Thus, a biomolecule separated from cells is considered to be substantially purified when separated from cellular components. Purified therefore does not require absolute purity. Furthermore, a “purified” biomolecule can include one or more other biomolecules. Thus, the term “purified” does not exclude combinations of biomolecules.

Substantial purity can be at least about 60% or more of the biomolecule by mass. Purity can also be about 70% or 80% or more, and can be greater, for example, 90% or more. Purity can be less, for example, the amount of a biomolecule by weight % can be less than 10% but the relative proportion of the biomolecule compared to other components with which it is normally associated with will be greater. Purity can be determined by any appropriate method, including, for example, mass spectrometry, UV spectroscopy, chromatography (e.g., HPLC, gas phase, paper), gel electrophoresis (e.g., silver or coomassie staining) and sequence analysis (peptide and nucleic acid).

In accordance with the invention, there are further provided methods for screening and identifying a compound (e.g., drug) or treatment that modulates binding of a biomolecule directly or indirectly to a region of interest of a nucleic acid in a cell. In one embodiment, a method includes providing a cell or plurality of cells (e.g., a sample of cells or tissue); contacting the cell or cells with a test compound or treatment; contacting or exposing the cell or cells to a fixing or cross-linking agent or treatment under conditions in which direct or indirect binding of one or more biomolecules to the nucleic acid is preserved or maintained, thereby producing fixed or cross-linked nucleic acid; optionally isolating the fixed or cross-linked nucleic acid; fragmenting the fixed or cross-linked nucleic acid, thereby producing fragmented fixed or cross-linked nucleic acid; optionally denaturing the fragmented fixed or cross-linked nucleic acid; hybridizing a capture oligonucleotide to the fragmented fixed or cross-linked nucleic acid to form a hybridized target, wherein the capture oligonucleotide is complementary to all or a part of the region of interest of the nucleic acid; isolating or purifying the hybridized target; identifying or characterizing one or more biomolecules directly or indirectly bound to the region of interest of the nucleic acid; and comparing binding of the one or more biomolecules to binding in the absence of the test compound. Altered (e.g., increased or decreased) binding in the presence of the test compound or treatment identifies the compound as a compound that modulates binding of the biomolecule to the region of interest of the nucleic acid in the cell.

Compounds that can be screened or identified according to the invention include drugs or therapeutic agents, and libraries of compounds such as peptides (antibodies, signaling molecules such as cytokines and chemokines) carbohydrates and organic molecules. Various libraries are available commercially or can be produced by methods known to the skilled artisan. For example, libraries of small synthetic molecules, prepared by combinatorial chemistry methods, are commercially available or can be produced by methods known to the skilled artisan.

The term “contacting,” when used in reference to a composition such as a cell material, sample, or treatment, means a direct or indirect interaction between the composition (e.g., cell) and the other referenced entity. A particular example of direct interaction is a physical interaction. A particular example of an indirect interaction is where the composition acts upon an intermediary molecule, which in turn acts upon the referenced entity. Thus, for example, contacting a cell with a fixing or cross-linking agent or treatment allows the fixing or cross-linking agent or treatment to fix or cross-link any biomolecules bound to nucleic acid in the cell.

The terms “assaying” and “measuring” and grammatical variations thereof are used interchangeably herein and refer to either qualitative or quantitative determinations, or both qualitative and quantitative determinations.

Cells applicable in the methods of the invention include but are not limited to prokaryotic and eukaryotic cells such as bacteria, fungi (yeast), plant, insect, and animal (e.g., mammalian, including primate and human) cells. The cells may be a primary cell isolate, cell culture (e.g., passaged, established or immortalized cell line), or part of a plurality of cells, or a tissue or organ ex vivo or in a subject (in vivo). Cells applicable in the methods of the invention include transformed cells. For example, bacteria transformed with recombinant bacteriophage nucleic acid, plasmid nucleic acid or cosmid nucleic acid expression vectors; yeast transformed with recombinant yeast expression vectors; plant cell systems infected with recombinant virus expression vectors or transformed with recombinant plasmid expression vectors (e.g., Ti plasmid); insect cell systems infected with recombinant virus expression vectors (e.g., baculovirus); and animal cell systems infected with recombinant virus expression vectors (e.g., retroviruses, adenovirus, vaccinia virus), or transformed animal cell systems engineered for stable expression.

The term “transformed” when use in reference to a cell or organism, means a genetic change in a cell following incorporation of an exogenous molecule, or mutation [by any means] of endogenous genomic nucleic acid, for example, introduction of a protein or nucleic acid (e.g., a vector or transgene) into the cell. Thus, a “transformed” cell is a cell into which, or a progeny thereof in which an exogenous molecule has been introduced by the hand of man, for example, by recombinant DNA techniques. Methods of the invention can employ such cells, regardless of whether the region of interest is located on an exogenously introduced nucleic acid (e.g., a vector or transgene) or an endogenous gene.

The invention further provides kits, including fixing and cross-linking agents and treatments, packaged into suitable packaging material, optionally in combination with instructions for using the kit components, e.g., instructions for performing a method of the invention. In particular embodiments, a kit includes a compound having nitrogen mustard and N-hydroxysuccinimide (NHS) ester, azide and N-hydroxysuccinimide (NHS) ester, or cis-platinum and N-hydroxysuccinimide (NHS) ester. In additional particular embodiments, a kit includes a combination of fixing or cross-linking treatments. In a particular aspect, a combination includes ethidium bromide monoazide and ethylene glycol bis-[succinimidyl succinate] (EGS), cis-platin and ethylene glycol bis-[succinimidyl succinate] (EGS), nitrogen mustard and ethylene glycol bis-[succinimidyl succinate] (EGS), and 4-aminomethyl trioxsalen and ethylene glycol bis-[succinimidyl succinate] (EGS).

The term “packaging material” refers to a physical structure housing the components of the kit. The packaging material can maintain the components sterilely, and can be made of material commonly used for such purposes (e.g., paper, corrugated fiber, glass, plastic, foil, ampules, etc.). The label or packaging insert can include appropriate written instructions, for example, practicing a method of the invention. The instructions may be on “printed matter,” e.g., on paper or cardboard within the kit, on a label affixed to the kit or packaging material, or attached to a vial or tube containing a component of the kit. Instructions may comprise voice or video tape and additionally be included on a computer readable medium, such as a disk (floppy diskette or hard disk), optical CD such as CD- or DVD-ROM/RAM, magnetic tape, electrical storage media such as RAM and ROM and hybrids of these such as magnetic/optical storage media.

Unless otherwise defined, all technical and scientific terms used herein have the same meaning as commonly understood by one of ordinary skill in the art to which this invention relates. Although methods and materials similar or equivalent to those described herein can be used in the practice or testing of the invention, suitable methods and materials are described herein.

All publications, patents, Genbank accession numbers and other references cited herein are incorporated by reference in their entirety. In case of conflict, the present specification, including definitions, will control.

As used herein, singular forms “a”, “and,” and “the” include plural referents unless the context clearly indicates otherwise. Thus, for example, reference to a “fixing or cross-linking agent or treatment” includes a plurality of fixing or cross-linking agents or treatments and reference to a method step can include performing the step multiple consecutive times, and so forth.

As used herein, all numerical values or numerical ranges include whole integers within or encompassing such ranges and fractions of the values or the integers within or encompassing ranges unless the context clearly indicates otherwise. Thus, for example, reference to a range of 90-100%, includes any numerical value or range within or encompassing such values, such as 91%, 92%, 93%, 94%, 95%, 95%, 97%, etc., as well as 91.1%, 91.2%, 91.3%, 91.4%, 91.5%, etc., 92.1%, 92.2%, 92.3%, 92.4%, 92.5%, etc., and any numerical range within such a range, such as 90-92%, 90-95%, 95-98%, 96-98%, 99-100%, etc.

The invention is generally disclosed herein using affirmative language to describe the numerous embodiments. The invention also specifically includes embodiments in which particular subject matter is excluded, in full or in part, such as substances or materials, method steps and conditions, protocols, procedures, assays or analysis. Thus, even though the invention is generally not expressed herein in terms of what the invention does not include, aspects that are not expressly included in the invention are nevertheless disclosed.

A number of embodiments of the invention have been described. Nevertheless, it will be understood that various modifications may be made without departing from the spirit and scope of the invention. Accordingly, the following examples are intended to illustrate but not limit the scope of invention described in the claims.

### EXAMPLES

### Example 1

This exampled includes a general description of an exemplary procedure outline.

Sample Source: Any cellular or viral sample type may serve as source material: cultured prokaryotic cells, cultured eukaryotic cells, an environmental sample, tissues, biological fluids, or lysates.

Fixation and Cross-Linking of Sample: Fixation or cross-linking of the sample in a cell or cells preserves the physical interaction between nucleic acids and biomolecules bound to them, such as proteins or other biomolecules, co-factors or pharmacological agents. Fixation or cross-linking preserves molecular complexes present on nucleic acids in vivo, and maintains the interactions through a stringent hybridization-based isolation procedure. Fixation or cross-linking can be achieved using chemical fixatives (agents) or cross-linkers or can be induced by a treatment such as exposure to radiation (e.g., ultraviolet or laser light). Fixation or cross-linking establish a covalent connection between nucleic acids and bound biomolecules. If desired, two or more (three, four, five, etc.) fixation or cross-linking agents or treatments may be employed to stabilize large molecular complexes on the nucleic acid, for example, by creating protein-protein bonds. (Nowak et al., BioTechniques. 2005; 39(5):715-25)

Samples May Be Fixed or Cross-Linked In Situ: cultured cells can be fixed in growth flasks in growth medium, and tissue samples may be immersed in a fixative or cross-linker. Alternatively, samples may be disrupted to facilitate efficient fixation or cross-linking. Disruption may be physical, such as grinding the sample; chemical, such as use of detergent to permeabilize the outer cell membrane; or enzymatic, such as use of trypsin, lysozyme, etc. to digest extracellular matrix proteins.

Fixative and Cross-linking Agents and Treatments: Any agent or treatment or combination of agents or treatments that preserve the physical connection between nucleic acid and bound biomolecules can be utilized as a fixative or cross-linker. Typically, the fixative or cross-linker establishes a covalent bond between the nucleic acid and one or more of the bound biomolecules.

Fixatives and cross-linkers can achieve covalent bonding between nucleic acid and one or more bound biomolecules through several mechanisms. For example, agents and treatments may alter the chemical structure of the nucleic acid or the bound biomolecules, such that they subsequently react spontaneously with each other. Examples of such agents and treatments include formaldehyde, dimethyl sulfate (the Mirzabekov protocol), sodium bisulfite, permanganate, hydrazine and carbodiimides (Chkheidze et al., FEBS Lett. 336:340 (1993)). An agent or treatment may form a chemical bridge, or crosslink, between the nucleic acid and bound biomolecules. In order to form a crosslink, an agent will typically include two or more functional groups: one which bonds covalently to the nucleic acid, and a second which bonds covalently to the bound biomolecules. Examples of functional groups which may bond covalently to nucleic acids include aldehyde-containing compounds such as glutaraldehyde and glyoxal (Kuykendall and Bogdanffy, Mutat Res. 283:131 (1992); aldehyde fixation followed by cross-link bond stabilization with reducing agents such as sodium cyanoborohydride or sodium borohydride (Kurtz and Lloyd, J Biol Chem. 278:5970 (2003); nucleic acid base alkylating agents such as nitrogen mustard derivatives and sulfur mustard derivatives (Baker et al., Mutat Res. November-December 132(5-6):171 (1984); nucleic acid backbone alkylating agents such as N-nitrosoureas and sulfonyl chlorides (Gonzaga et al., Nucleic Acids Res. 18:3961 (1990); groups containing reactive halogens, such as N-bromo-succinimide; nucleic acid adduct-forming metallic compounds such as cis-platinum, trans-platinum and other platinum derivatives, potassium chromate, hexavalent chromium, organometallic compounds such as organometallic copper compounds (Costa et al., Mutat Res. 369:13 (1996); epoxide groups such as butadiene diepoxide; and photoactive groups such as azides, psoralen, and trioxsalen.

Examples of functional groups which may bond covalently to biomolecules bound to nucleic acids include amine-specific reactive groups such as N-hydroxy succinimide, imidoesters, hydroxymethyl phosphine, nitrogen mustards, sulfur mustards, (Chiaruttini et al., Nucleic Acids Res. 1982; 10(23):7657-76); sulfhydryl-specific reactive groups such as maleimide, vinylsulfone and pyridyl disulfide; hydroxyl-specific reactive groups such as isocyanate; carbohydrate-specific reactive groups such as hydrazine; and broadly reactive groups such as azides (including photoactive azides), epoxides, and organometallic groups such as cis-platinum.

Reactive groups can optionally be targeted to the nucleic acid by including groups with high chemical affinity for nucleic acids such as intercalators (e.g., ethidium bromide, propidium iodide, angelicin, aziridine, quinacrine and anthracyclines), nucleic acid duplex groove-binding groups (e.g., Hoechst 33258, Hoechst 33342, 4′,6-diamidino-2-phenylindole and lexitropsins), and phosphate backbone interacting groups (e.g., spermine, polyethylene imine and quaternary ammonium.)

In a combination crosslinking process, one type of bifunctional agent may bond to the nucleic acid and in the process label it with a new functional group A, while a separate bifunctional agent bonds with associated factors and with functional group A, forming a single chemical crosslink from multiple fixative agents. Such combination crosslinking may utilize agents composed of any combination of the functional groups described herein.

In a tissue culture setting, reactive agents may be incorporated into a growth medium of a cell or tissue sample, allowing incorporation of fixing or cross-linking agents into the biological structures themselves in vivo. For example, if a sample is grown in the presence of the photoactive biocompatible agent 5-bromo-deoxyuridine, the fixative may be incorporated into sample nucleic acid polymer structures, facilitating instant fixation by subsequent exposure to activating radiation. Photoleucine or photomethionine may similarly be used to incorporate activatable fixatives into bound factors.

Fixatives and crosslinking agents used may be labeled with detectable markers or tags, such as isotopes or electron spin-labels to facilitate tracking through isolation or purification or to allow quantitative comparisons of end analysis results.

Covalent bonds between nucleic acid and bound biomolecules can also be formed by exposure to ultraviolet radiation (Pashev et al., Trends Biochem Sci. 16:323 (1991); and Olinski et al., Chem Biol Interact. 34:173 (1981)). Exposure to UV, light radiation or laser radiation can also be used alone or in combination with chemical fixatives or cross-linkers to activate photoactive fixative groups.

Nucleic Acid Complex Isolation and Fragmentation: After fixation or cross-linking, biomolecule bound nucleic acid is harvested by lysis of cells. Lysis can be achieved by a number of methods including mechanical disruption or chemical treatment, such as freeze-thaw cycles, mechanical homogenization, disruption in a French press, hypotonic lysis, alkaline lysis, sonication, use of detergents, use of chaotropic salts, combinations thereof, and other standard cell lysis methods. Alternatively, nuclei (or other subcellular compartments, such as mitochondria, other organelles, or viral particles, etc.) of fixed eukaryotic cells may first be isolated away from other cellular material before they are lysed. If desired, the bound nucleic acids may be further purified from cellular contaminants after lysis. Optional isolation and purification methods include clarification by centrifugation, buffer exchange, or nucleic acid-selective fractionation of the lysate.

To physically separate nucleic acid, nucleic acids can be fragmented. Fragmentation methods developed for use in chromatin immunoprecipitation protocols can be employed. Nucleic acids may be fragmented by mechanical means (such as sonication or shearing forces such as that achieved with syringe-passage), by nucleases (specific restriction endonucleases or non-specific nucleases), by targeted enzyme cleavage (e.g., RARE {RecA (recombinase A) assisted restriction endonuclease cleavage} or triplex-assisted cleavage methods) or by chemical cleavage (Fors et al., Pharmacogenomics. 1:219 (2000); Elsner and Lindblad, DNA. 8:697 (1998); O'Neill and Turner, Methods. 31:76 (2003); Lefevre and Bonifer, Methods Mol Biol. 325:315 (2006)).

Generation of Locus-Specific Capture Oligonucleotides: Nucleic acid hybridization probes are a means by which desired genomic nucleic acid is isolated from the all genomic sequences in a cell or tissue sample. In practice, any nucleic acid functional analogue that exhibits Watson-Crick base-pairing or Hoogsteen (triplex) bonding may be used for capture oligonucleotide synthesis: oligodeoxyribonucleotides, oligoribonucleotides, duplex DNA (e.g., products of a polymerase chain reaction), RNA strands, nucleic acid from a natural source (e.g., a plasmid, virus genome, or genomic restriction fragment), locked nucleic acids, peptide nucleic acids (PNA), lexitropsins, or any other DNA base analogue or binding agent that allows sequence-specific hybridization (Millar et al., Anal Biochem. 226:325 (1995); Nielsen, Curr Opin Biotechnol. 12:16 (2001); Vester and Wengel, Biochemistry. 43:13233 (2004)).

Capture oligonucleotide sequences are designed to at least partially complementary—that is, capable of forming specific nucleic acid base pairing interactions—to nucleic acid that includes the region of interest. Design guidelines for probe length, melting temperature, and base composition may be adopted from in situ hybridization techniques (Jain K K, Med Device Technol. 15:14 (2004); King et al., Mol Diagn. 5:309 (2000). Several capture oligonucleotides complementary to all or a portion of the region of interest may be used as part of a single isolation protocol.

Capture oligonucleotides may be immobilized on a substrate or covalently tagged or modified with a detectable moiety or label that facilitates purification or detection of the hybridization complex. For example, a capture oligonucleotide may be immobilized on a substrate (such as a nylon or cellulose or other type of membrane or glass slide). Multiple capture oligonucleotides may be immobilized in different locations on the same substrate to facilitate investigation of multiple regions of interest. A tag, such as an affinity tag (e.g., biotin, avidin, etc.) may be conjugated to the capture oligonucleotide allowing purification of the complex on immobilized avidin or biotin substrates.

Purification Via Hybridization: The fixed or cross-linked fragmented sample nucleic acid is allowed to come into contact with a capture oligonucleotide under conditions that facilitate nucleic acid sequence-specific hybridization. A fixed or cross-linked sample can first be denatured, such as brief heating to a temperature of at least 70° Celsius or chemical means such as alkaline treatment at a pH of greater than about 9. Denaturation can be omitted, for example, if a hybridization-promoting agent, such as the E. coli Recombinase A (recA) protein, is incorporated, or if favorable probe binding kinetics allows a passive hybridization approach (Rice et al., Genome Res. 14:116 (2004). A hybridization-promoting agent, such as the E. coli recA protein, can therefore be used to facilitate hybridzation such that denaturation is optional.

The nucleic acid/bound biomolecule complex is brought to a target hybridization temperature, as determined to be suitable for a particular sequence or sample, and incubated. Exact hybridization temperatures, times, and buffer compositions are determined by the nature of the sample, the type of fixative agent or cross-linking, and the melting temperature of the nucleic acid probe. Nonspecific blocking agents, such as natural or synthetic polynucleotides, may be included in the hybridization. Agents that alter nucleic acid hybridization activity, such as dimethyl sulfoxide, formamide, dimethyl formamide, betaine, or other agents, also can be added to the hybridization mixture.

During hybridization, the fixed or cross-linked nucleic acid/biomolecule complex from the region of interest binds specifically to the capture oligonucleotide. At the conclusion of hybridization, the desired nucleic acid/biomolecule complexes now bound to the capture oligonucleotide, are physically separated from the remainder of the hybridization solution containing undesired sample complexes. Separation can be achieved by removing unbound (unhybridized) material from an immobilized substrate (as above) to which the capture oligonucleotide is attached. Additional contaminants may be removed by washing the bound material with a buffer.

Biomolecule Recovery: Fixed or cross-linked nucleic acid/biomolecule complexes may be separated, if desired, from a capture oligonucleotide by a variety of techniques, such as thermal, chemical, or mechanical disruption. For simple integration with subsequent steps, fixed or cross-linked nucleic acid/biomolecule complex may be dissociated by incubation in a buffer under denaturing conditions (Hultman et al., Nucleic Acids Res. 17:4937 (1989)). Following denaturation, the purified Fixed or cross-linked nucleic acid/biomolecule complex is removed from the matrix and may be analyzed as desired. Alternatively, fixed or cross-linked nucleic acid/biomolecule may be analyzed directly on the matrix, such as in situ MALDI (Matrix-assisted Laser Desorption Ionization) Mass Spectroscopy or other method for determining molecular characteristics.

The method employed for recovery of biomolecule(s) from fixed or cross-linked nucleic acid/biomolecule complexes may be determined by the fixation/cross-linking method and the intended analysis technique. If the sample was fixed or cross-linked with a reversible agent, such as formaldehyde, the purified complex may simply be incubated in conditions that reverse the cross-links, such as heating to greater than 50° C. for at least about two hours, If the cross-linking is more difficult to reverse, fragments of individual binding biomolecule constituents may be freed from the complex through chemical cleavage. For example, trypsin can liberate peptide fragments from a complex that may be immediately analyzed by mass spectrometry (Geyer et al., Nucleic Acids Res. 32:e132 (2004)). Alternatively, cross-link reversal may be omitted and the entire fixed or cross-linked nucleic acid/biomolecule complex may be analyzed by fragmentation in a mass spectrometry instrument or other apparatus suitable for evaluation of molecular components (Muller et al., Eur J Biochem. 268:1837 (2001)).

Bound biomolecules may also be liberated and/or recovered simply by degradation of the nucleic acid. Examples of such methodology include formic acid hydrolysis and (exo)nuclease digestion. These methods may leave behind small chemical adducts at the point of nucleic acid-bound biomolecule crosslink formation which can be taken into account during analysis. Such adducts may be useful as indicators of nucleic acid-bound biomolecule contact points, thus providing additional molecular structure or binding site information (Synowsky et al., Mol Cell Proteomics. 5:1581 (2006)).

Post-Recovery Analysis: After simple preparative work up (buffer exchange, concentration, sample transfer, etc.), isolated or biomolecule(s) purified can be subject to analysis techniques including, but not limited to, gel electrophoresis, western blotting, gel shift analysis, mass spectrometry, chromatography, nuclear magnetic resonance (NMR), crystallography, sequencing or other methods. The presence of unique post translational modification sites (for example unique phosphorylation, acetylation, methylation, nitration, glycosylation, etc., sites on a biomolecule) can also be determined—the method will allow isolation of all of the differentially modified biomolecules without prior knowledge of the state of phosphorylation, acetylation, methylation, nitration, glycosylation, etc.

### Example 2

The following protocol is a specific method of the invention. In this example, a plasmid containing the lac repressor DNA binding sequence is purified from formaldehyde-crosslinked bacterial whole lysate. Bound lac repressor protein is recovered from the purified sample, but not from the negative control sample.

Materials
Frozen glycerol stock of Escherichia coli strain (DH5α, New England Biolabs) harboring target plasmid (pBS SK+, Stratagene)
2YT Medium: 10 g tryptone (Sigma), 5 g NaCl (Sigma), 5 g yeast extract (Sigma) per liter, sterile)
Ampicillin stock solution (100 g/L) (Sigma)
Formaldehyde solution (37% vol/vol) (Sigma)
TEN Buffer: 10 mM Tris (Sigma), 1 mM EDTA (Sigma), 150 mM NaCl (Sigma), sterile
Sterile nuclease- and protease-free water
500,000 units/mL lysozyme stock solution (Sigma)
Bacterial Protease Inhibitor Cocktail (AEBSF, E-64, pepstatin A, EDTA, bestatin) (Sigma)
Rnase I (Ambion)
Sca I Restriction Endonuclease (New England Biolabs)
10% SDS solution (mass/vol) (Sigma)
19 gauge needle and sterile syringe (Baxter)
Synthetic 5′ biotinylated oligonucleotides complementary to target DNA region (100 μM in water) (IDT, Inc.)
Synthetic 5′ biotinylated control oligonucleotides (100 μM in water) (IDT, Inc.)
Streptavidin-conjugated magnetic beads (binding capacity >400 pmol biotin per mg) (Novagen)
Magnetic separation stand (Novagen)
Phosphate Buffered Saline (PBS): 150 mM NaCl (Sigma), 8.1 mM Na₂HPO₄ (Sigma), 1.9 mM
NaH₂PO₄ (Sigma), pH 7.4, sterile
Salmon sperm DNA (Sigma)
10N NaOH (Sigma)
0.15N NaOH (Sigma)
20×SSC Buffer: 3M NaCl (Sigma), 0.3M Sodium Citrate (Sigma), pH 7.0, sterile
2×SSC Buffer: 0.3M NaCl (Sigma), 0.03M Sodium Citrate (Sigma), pH 7.0, sterile
Formamide (Sigma)
50× Denhardt's Solution (1% mass/vol each of bovine serum albumin, Ficoll, and polyvinylpyrrolidone) (Sigma)
Glacial acetic acid (Sigma)
1 M Tris (Sigma) pH 7.0
2×CR Buffer: 2% SDS (Sigma), 500 mM 2-mercaptoethanol (Sigma), 0.2M sodium bicarbonate (Sigma)

In brief, a scrape of frozen glycerol stock culture of Escherichia coli strain DH5α harboring plasmid pBS SK+ was inoculated into 500 mL 2YT Medium supplemented with 100 mg/L ampicillin. The culture was grown with shaking at 37° C. for 18 hours until reaching a cell density of 2.0×10⁹ cells/mL, for a total cell count of approximately 2.0×10¹². Two cultures (test and negative control samples) were prepared and subjected to identical treatment throughout the protocol except for the capture oligonucleotides used for isolation. Formaldehyde was added to the culture to a final concentration of 1% (vol/vol). Formaldehyde-treated cells were incubated at 4° C. for 18 hr. Formadehyde-treated cells were harvested by centrifugation at 4000 rpm in a tabletop centrifuge at 4° C., followed by removal of the supernatant. Pelleted cells were resuspended in 100 mL ice-cold TEN Buffer by vortexing and re-pelleted at 4000 rpm in a 4° C. centrifuge. This wash was repeated four times. Washed, pelleted cells were frozen at −80° C. until further processing.

Frozen pelleted cells were thawed on ice. Thawed pellets were resuspended by vortexing in a solution of 750,000 units lysozyme and 500 μL bacterial protease inhibitor cocktail in a total volume of 2 mL. To linearize plasmid, 1000 units of Sca I restriction endonuclease was added, and cellular RNA was degraded by addition of 1000 units of RNase I. This mixture was incubated at room temperature for 2 hours. 10% SDS stock was added (˜30 μL) to a final concentration of 0.1%. After thorough mixing, lysis proceeded at room temperature for an additional 30 minutes. Cells were further disrupted by passage through a 19 gauge needle and syringe five times. Cell lysate was centrifuged at 10,000 rpm to remove debris, with the clarified supernatant retained for further manipulation.

Capture oligonucleotides were designed to contain base sequences complementary to regions of target plasmid. Capture oligonucleotides were synthesized with a biotin moiety covalently attached at the 5′ end to facilitate binding to streptavidin-conjugated magnetic beads. The capture oligonucleotide used in the test sample first hybridization step, referred to as pBS.H1, had the following sequence: 5′-biotin-TGA GGG TTA ATT GCG CGC TTG GCG TAA TCA TGG TCA TAG C-3′ (SEQ ID NO: 1). The capture oligonucleotide used in the test sample second hybridization step, referred to as pBS.H2, had sequence: 5′-biotin-TTG TAA AAC GAC GGC CAG TGA GCG CGC GTA ATA CGA CTC AC-3′ (SEQ ID NO:2).

For the negative control sample, oligonucleotides were not complementary to a sequence on the plasmid, but had the same overall base composition. The oligonucleotide used in the negative control sample first hybridization step, referred to as pBS.N1, was a scrambled version of the pBS.H1 sequence: 5′-biotin-GGC CAG TGG TTG TGG AAC GGT ATA CTT CAT GTA AGC GTT C-3′ (SEQ ID NO:3). The oligonucleotide used in the negative control sample second hybridization step, referred to as pBS.N2, was a scrambled version of the pBS.H2 sequence: 5′-biotin-GAG TTC CGG AGC TGG CCA CAA ACC AGA GAA TCT CTA TCG AG-3′ (SEQ ID NO:4).

Capture and control oligonucleotides dissolved in sterile water at a concentration of 100 μM were denatured by heating to 95° C. for 10 minutes, followed by immediate cooling on ice. A total of 2 nanomoles of oligonucleotide were incubated at room temperature with 4 mg of streptavidin-conjugated magnetic beads, with the reaction diluted to a total volume of 5 mL with PBS. The reaction continued for 1 hour with gentle agitation. Oligonucleotide-bead conjugates were then isolated from unbound material by incubation in a magnetic stand for 5 minutes. Supernatant was removed and discarded. The beads were washed three times in PBS, with the isolation procedure repeated each time. Oligonucleotide-conjugated beads were finally resuspended in 500 μL PBS.

For hybridization of clarified cell lysate (1 mL) was mixed with 1 mg sheared salmon sperm DNA (Sigma) in a total volume of 2 mL. The mixture was denatured by addition of 10N NaOH to a final concentration of 0.15N, with a pH of 12.5. The denaturation step proceeded at room temperature for 30 minutes. 1 mL of 20×SSC buffer, 5 mL formamide, and 500 mL 5× Denhardt's Solution, and the pBS.H1 or pBS.N1 oligonucleotide-conjugated magnetic beads were added, and the mixture incubated at room temperature for an additional 15 minutes. The mixture was then titrated to pH 7.5 with glacial acetic acid and brought to 10 mL total volume with sterile water. This hybridization mix was incubated at room temperature with agitation for 16 hours. Following hybridization, the bead-bound complexes were isolated by incubation in a magnetic stand for 5 minutes, with subsequent removal of supernatant. The beads were washed 3 times for 10 minutes each in 2×SSC, with magnetic isolation carried out after each step as described above. Hybridized DNA-protein complexes were eluted from the magnetic bead/oligonucleotide substrate by denaturation in 0.15N NaOH (pH 12.5) for 15 minutes at room temperature. This denaturation process is strong enough to disrupt DNA base-pairing, but does not cause dissociation of biotin-avidin complexes. Oligonucleotide-linked beads were separated from the eluate by incubation for 5 minutes in the magnetic stand, followed by removal of the eluate.

Denatured eluate was mixed with 1 mg sheared salmon sperm DNA and incubated at room temperature for 30 minutes. 1 mL of 20×SSC buffer, 5 mL formamide, and 500 mL 50× Denhardt's Solution, and the pBS.H2 or pBS.N2 oligonucleotide-conjugated magnetic beads were then added. The mixture was incubated at room temperature for an additional 15 minutes. The mixture was then titrated to pH 7.5 with glacial acetic acid and brought to 10 mL total volume with sterile water. The hybridization mixture was again incubated for 16 hours at room temperature with agitation. Beads and bound complexes were isolated and washed three times as described above.

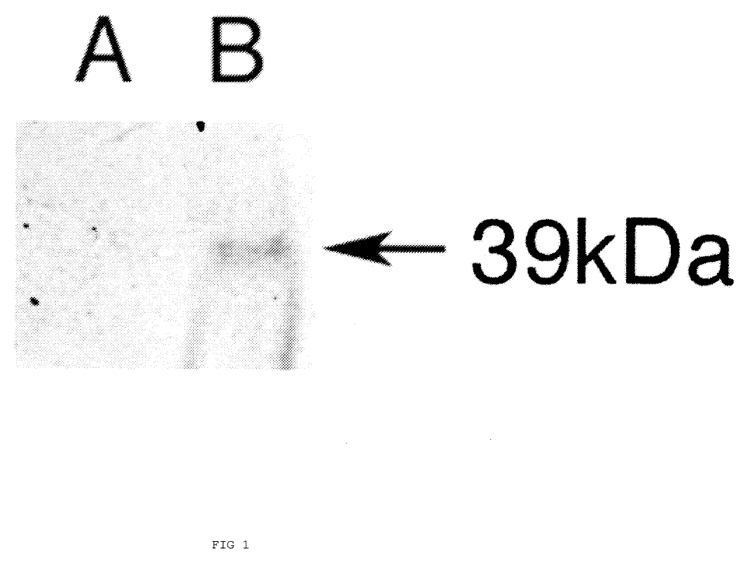

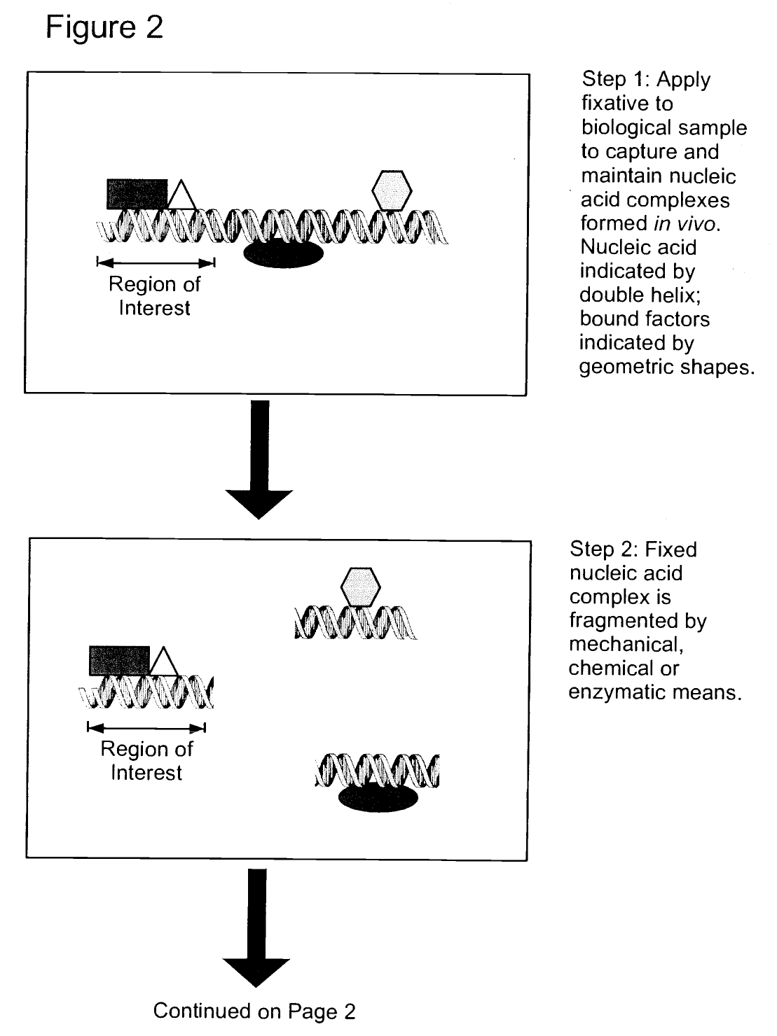

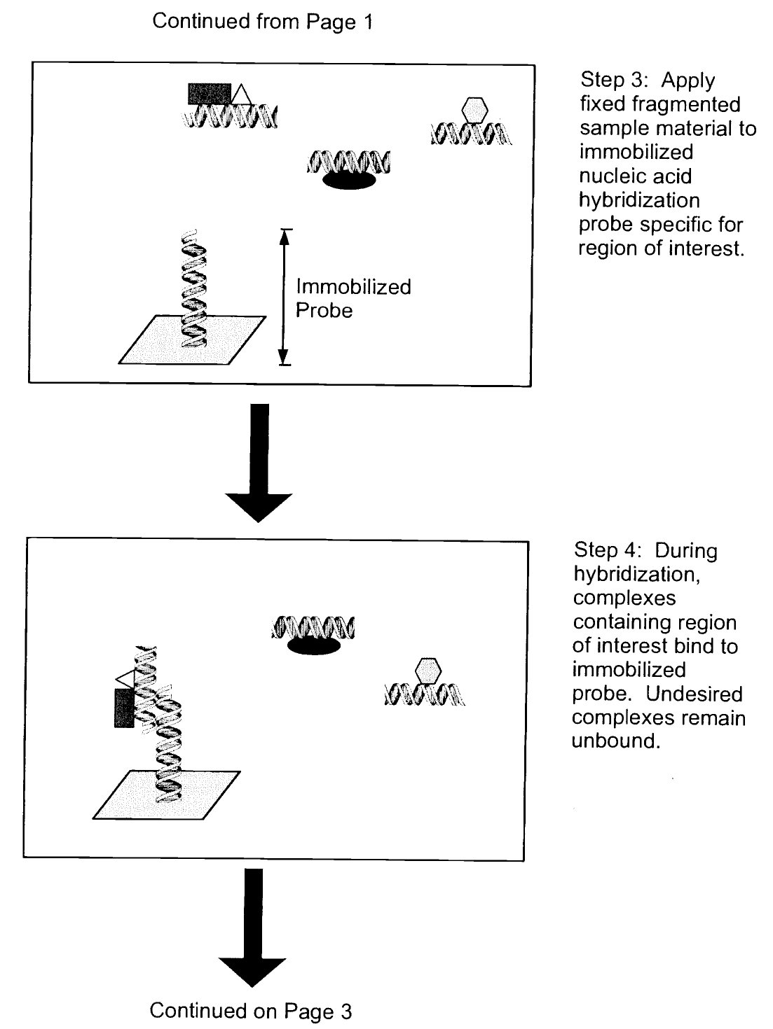

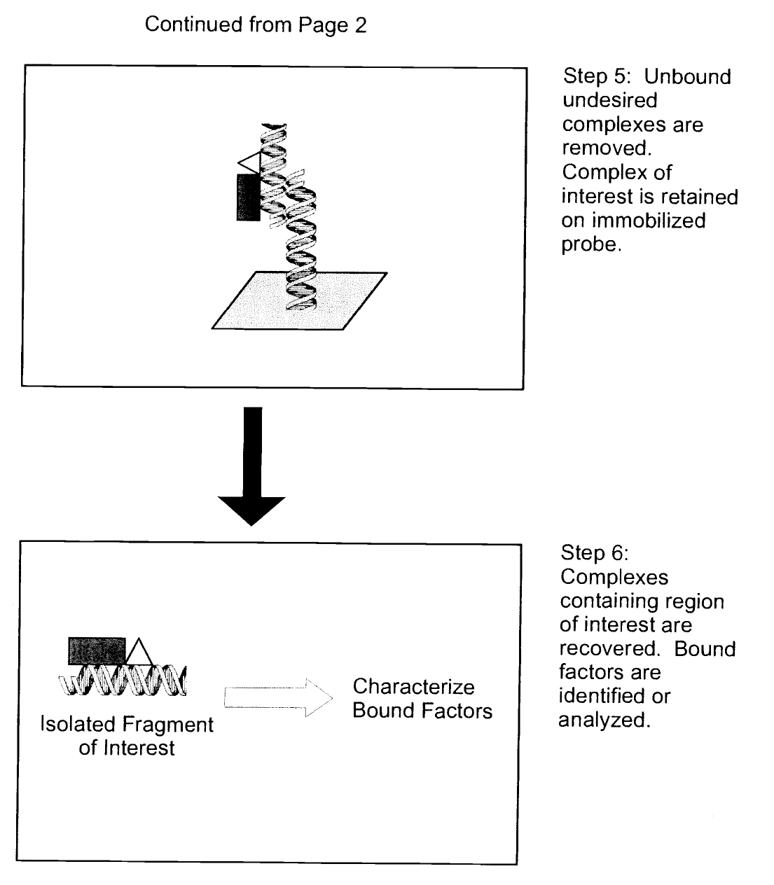

Following the final hybridization washes, the bound material was eluted from the beads by denaturation in 50 μL of 0.15N NaOH (pH 12.5) for 15 minutes at room temperature. Beads were separated by incubation for 5 minutes in a magnetic stand, and the supernatant was isolated. The supernatant was neutralized by addition of 50 μL of 1 M Tris pH 7.0. To reverse cross-links, 100 μL of 2×CR Buffer was added and the material was incubated at 65° C. for 24 hours. Following cross-link reversal, 25 μL aliquots of each sample (control and experimental) were run on a 10% denaturing acrylamide gel for 1 hour at 100 volts and visualized using the Invitrogen Silverquest protein staining kit (FIG. 1).

### Example 3

The following example includes a description of particular non-limiting applications of the invention.

In accordance with the invention, analysis of molecular complexes at any desired nucleic acid site (e.g., genomic site) in a cell, based solely on knowledge and selection of nucleic acid sequence at the site of interest, can be performed. Particular examples include:

Analysis Targeting DNA Sequences and Biomolecules that Bind DNA: Promoter analysis; transcriptional termination factors at specific sites; factors binding to special DNA structures, e.g. palindromes, Z-DNA, single-stranded loop regions, G-quadruplexes; analysis of chromosomal looping and associated binding factors (intra- or inter-chromosomal); analysis of epigenetic factors such as DNA methylation or other covalent modification, histone acetylation or deacetylation, histone subunit composition and stoichiometry; elucidation of transcriptional complexes associated with any in vivo DNA structure, whether chromosomal or extra-chromosomal replicons (any episomal replicon including plasmids, F factors and viral episomes; mobile elements, mitochondria; chloroplasts; kinetoplasts; micronuclei, etc.); DNA replication initiation sites; and DNA locus-specific mitotic machinery (proteins bound at centrosomes during mitosis, etc.)

Analysis Targeting RNA Sequences and Biomolecules that Bind RNA: Regulatory factors binding to particular mRNA structures/sequences, including low-molecular weight compounds acting as riboswitch modulators; splicing factors; factors associated with nonsense-mediated mRNA decay; factors binding to 3′ sequences modulating mRNA stability/half-life; factors associated with 3′ end formation of mature mRNAs; factors associated with RNA (mRNA, rRNA, tRNA) processing and nuclear export; and factors associated with the iRNA machinery (e.g. action of specific miRNAs).

In general, biomolecules bound to isolated or purified nucleic acid loci can identify biomolecules bound to or associated with those loci in a cell. Such biomolecules include, but are not limited to, the following: Proteins; any associated nucleic acid, irrespective of covalent modifications or structural isomerization; synthetic or naturally-derived compounds and small biomolecules, such as drugs; hormones (including proteins) or their analogs; vitamins or metabolic co-factors; inorganic atoms, ions, molecules, or complexes; lipids, fats, waxes, oils, or their derivatives; small messenger biomolecules or metabolites, such as cyclic AMP and glucose; solvent molecules; complexes of any combination of the foregoing; and functionally-derivatized analogs of the foregoing (e.g., non-hydrolyzable metabolites, or cross-linking enabled drug analogs).

Non-limiting particular examples of clinical and diagnostic applications include:

Tumor and Cancer Diagnostic Applications: Classifying tumor subsets by means of transcription factor or other DNA-binding protein profiles.

Model and Background Information: Tumor and cancer subsets differ in various DNA or RNA binding activities, either up- or down-regulated. If clinically significant, it is likely that the consequences of these aberrations are manifested through certain key genes (X, Y, Z, etc) upon which the DNA or RNA-binding biomolecules act. In this model, information from previous work has revealed the significant proteins which bind to the regulatory regions of key genes X, Y, Z in the normal state. The model assumes that specific tumor and cancer cell subsets can be distinguished by the binding patterns at specific genomic regulatory sites. In accordance with the invention, genetic loci in a tumor or cancer cell can be probed for differences in biomolecule binding among tumor and cancer cell subsets.

Genetic, Autoimmune, Infectious Disease, Pharmacological and Other Diagnostic Applications: Detection and diagnosis of clinical samples for genetic Disorders including pre-natal diagnostics; diabetes and early detection; autoimmune disorders and allergic responses; infectious agent (viral, bacterial, fungal) detection; and drug reactions, toxicity and efficacy.

### Example 4

The following example includes a description of particular non-limiting fixative and cross-linking agents and treatments.

“NHS ester” refers to the N-hydroxysuccinimide functional group. “EGS” refers to the homobifunctional amine-amine crosslinking molecule (ethylene glycol bis-[succinimidyl succinate.])

### Compounds Synthesized:

| | | Protein-binding | |
|---|---|---|---|
| Compound | DNA-binding moiety | moiety | Activity |
| A | Nitrogen mustard | NHS ester | Strong |
| B | Azide (photoactive) | NHS ester | Strong |
| C | Cis-platinum | NHS ester | Some |
| D | Psoralen (photoactive) | NHS ester | None |
| | | | observed |
| E | Epoxide | NHS ester | None |
| | | | observed |

### Combination Crosslinking:

| First agent (DNA- | Second agent (amine | |
|---|---|---|
| binding) | binding) | Activity |
| Ethidium bromide | EGS | Strong |
| monoazide (photoactive) | | |
| Cisplatin | EGS | Strong |
| Nitrogen mustard (Mirus) | EGS | Strong |
| 4-aminomethyl trioxsalen | EGS | Weak |
| (photoactive) | | |

Additional fixative agents that showed some activity include formaldehyde; glutaraldehyde; glyoxal; quinacrine mustard; and 1,3 butadiene diepoxide.

Note: For Combination crosslinking experiments, both the First and Second agents are added more or less simultaneously to whole cells (ethidium bromide requires prior membrane permeabilization) for not less than 30 minutes at room temperature to 37 Celsius.

Several cross-linking fixatives incorporating a nucleic acid binding group covalently coupled with a primary amine-binding group were synthesized. These agents were assayed to determine effectiveness covalently linking nucleic acids to a bound factor.

Cross-Linking Compound 1: Nitrogen mustard group with an N-hydroxy-succinimidyl primary amine-binding group.
3-(4-(bis(2-chloroethyl)amino)phenyl)-2-(4-(2-(4-(2,5-dioxopyrrolidin-1-yloxy)-4-oxobutanoyloxy)ethoxy)-4-oxobutanamido)propanoic acid
Chemical Formula: C₂₇H₃₃Cl₂N₃O₁₁
Molecular Weight: 646.47

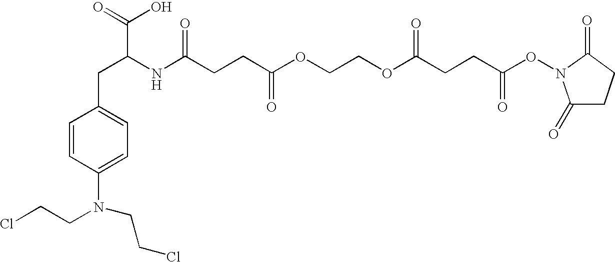

This agent incorporates a nitrogen mustard nucleic acid binding group with an N-hydroxy-succinimidyl primary amine-binding group. It is capable of rapidly cross-linking nucleic acids to amine-containing bound factors in a single step, and it is cleavable under mild conditions (pH 8.5 in the presence of hydroxylamine at 37° C.) if cross-link reversal is desired. This agent is exemplary of a class of cross-linking fixatives incorporating a nitrogen mustard nucleic acid binding group as well as an N-hydroxy-succinimidyl amine-biding group.

Synthesis was carried out by mixing equimolar quantities of melphalan (4-[Bis(2-chloroethyl)amino]-L-phenylalanine) (Sigma Aldrich, product # M2011) and ethylene glycol bis-(succinimidyl succinate) (Pierce, product # 21565) at room temperature in dimethyl sulfoxide for 30 minutes. The synthesized product was used directly in cross-linking assays.

Cross-Linking Compound 2: Photoactive azide coupled to a nucleic acid intercalator and and an N-hydroxy-succinimidyl primary amine-binding group.
8-azido-3-(4-(2-(4-(2,5-dioxopyrrolidin-1-yloxy)-4-oxobutanoyloxy)ethoxy)-4-oxobutanamido)-5-ethyl-6-phenylphenanthridinium bromide
Chemical Formula: C₃₅H₃₃BrN₆O₉
Molecular Weight: 761.58

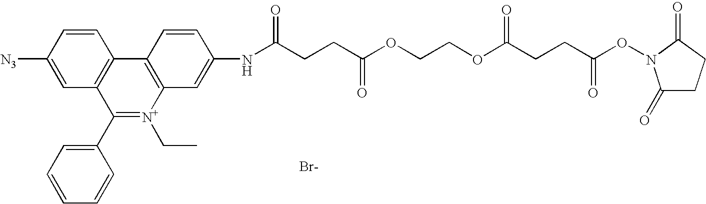

This agent incorporates a photoactive azide coupled to a nucleic acid intercalator as the nucleic acid binding group, and an N-hydroxy-succinimidyl primary amine-binding group. Incorporation of intercalator increases affinity of the agent for nucleic acid, increasing the cross-linking efficiency of the agent. The agent is activated by a light source (visible light is sufficient) to undergo cross-linking. It is cleavable under mild conditions (pH 8.5 in the presence of hydroxylamine at 37° C.) if cross-link reversal is desired. This compound is exemplary of a class of photoactive azide agents targeted specifically to nucleic acid, also including an amine-biding group and a cleavable chain.

Synthesis was carried out by mixing equimolar quantities of ethidium bromide monoazide (3-Amino-8-azido-5-ethyl-6-phenylphenanthridinium bromide) (Sigma product # E2028) with ethylene glycol bis-(succinimidyl succinate) (Pierce, product # 21565) at room temperature in dimethyl sulfoxide for 30 minutes in the dark. The synthesized product was used directly in cross-linking assays.

Cross-Linking Compound 3: Divalent Platinum Derivative+N-hydroxy-succininimidyl amine binding group.
2,5-dioxopyrrolidin-1-yl-2-(4-(3-methylthioureido)-4-oxobutanoyloxy)ethyl succinate chloro(ethylenediamine)platinum(II) complex
Chemical Formula: C₁₈H₂₉ClN₅O₉PtS
Molecular Weight: 722.05

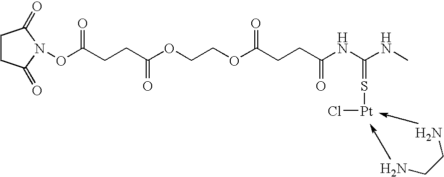

This agent incorporates a divalent platinum derivative as a nucleic acid binding group and an N-hydroxy-succinimidyl amine-binding group. It is cleavable under mild conditions (pH 8.5 in the presence of hydroxylamine at 37° C.) if cross-link reversal is desired. This agent is exemplary of a class of cross-linking fixatives utilizing platinum derivatives as nucleic acid binding groups with N-hydroxy-succinimide as amine-binding groups.

Synthesis was carried out by first mixing a 1.5 molar excess of ethylene glycol bis-(succinimidyl succinate) (Pierce, product # 21565) with N-methylthiourea (Sigma, product # 84607) in dimethyl sulfoxide for 30 minutes at room temperature. The resulting product was mixed with dichloro(ethylenediamine)platinum(II) (Sigma, product # 244929) in a molar amount equal to the starting N-methylthiourea amount. This reaction was carried out in 50% dimethyl sulfoxide 50% water solution for 15 minutes. The synthesized product was used directly in cross-linking assays.

Cross-Linking Assays

The fixative agents were studied for cross-linking efficacy in a gel-shift assay. The Saccharomyces cerevisiae GAL4 protein and GAL4 deoxyribonucleic acid target sequence were used as a representative complex of nucleic acid and bound factor.

Materials:

Recombinant purified Saccharomyces cerevisiae GAL4 protein (Santa Cruz Biotechnology, product # sc-4050)
GAL4 target sequence oligonucleotide duplex (Integrated DNA Technologies):

Top strand sequence (SEQ ID NO: 5):
5′-GATCCAATACGACTCACTATAGCGGAAGACTCTCCTCCGGAGG-3′
Bottom stand sequence (SEQ ID NO: 6):
5′-GATCCCTCCGGAGGAGAGTCTTCCGCTATAGTGAGTCGTATTG-3′

1×PBS Buffer: 137 mM NaCl, 2.7 mM KCl, 10 mM Na₂HPO₄, 2 mM KH₂PO₄. pH 7.4
Loading Dye: 10 mM Tris-Hcl, 0.03% bromophenol blue, 50% glycerol, 10 mM EDTA
0.5×TBE Buffer: 0.5 Molar Tris-HCl, 0.5 Molar sodium borate, 0.25 Molar EDTA
8% Polyacrylamide gel prepared by standard methods in 0.5×TBE, 8 cm×10 cm×1 mm
Electrophoretic Mobility Shift Assay Kit (Invitrogen, product # E33075)

### Method:

In standard 500 microliter microcentrifuge tubes, 500 nanograms of GAL4 protein and 1 picomole of GAL4 target oligonucleotide duplex were mixed in a total volume of 25 microliters 1×PBS buffer and incubated at room temperature for 15 minutes. One tube for each novel cross-linking agent was prepared, as well as a negative control tube to which was not treated with cross-linker. Synthesized cross-linkers described above were added to the mixtures to a final concentration of 2 mM. Two microliters of 1×PBS buffer was added to the negative control reaction. Cross-linking reactions were allowed to proceed for 30 minutes at room temperature. The tube containing Novel Cross-linking Fixative 2 was incubated in the dark except for the final 5 minutes of the incubation, when it was placed in direct sunlight.

At the end of the cross-linking incubation, reactions were heated to 95° C. on a heat block for 10 minutes to denature non-covalently bound GAL4 protein and dissociate it from the oligonucleotide duplex. Loading dye (3 microliters) was then added to each tube and entire reactions were loaded onto an 8% polyacrylamide gel in 0.5×TBE buffer. Electrophoresis of the gel was carried out in 0.5×TBE buffer for 45 minutes at a constant voltage of 150 volts.

At the conclusion of electrophoresis, nucleic acids in the gel were fluorescently stained using the Invitrogen Electophoretic Mobility Shift Assay Kit according to the standard protocol. The stained gel was visualized under ultraviolet illumination on a Bio-Rad gel documentation system.

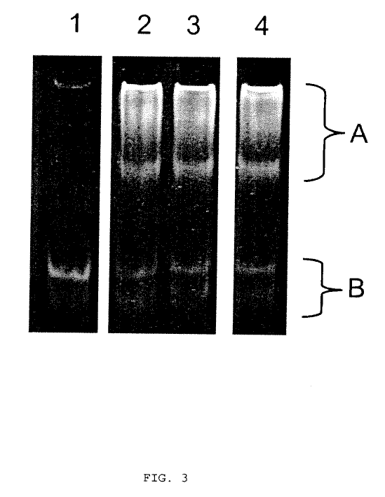

FIG. 3 illustrates the gel shift assay results. Free nucleic acid duplex migrates more quickly in the gel, while complexes of nucleic acid and bound factors show retarded migration. Since the reactions were denatured before loading, the negative control containing no cross-linking fixative shows little if any retardation of nucleic acid migration. Each of the reactions treated with a the aforementioned cross-linking agent shows significant retardation of nucleic acid migration, indicating that the complexes were covalently linked, and therefore not dissociated by denaturation. Each of the foregoing cross-linking agents synthesized effectively forms cross-links between nucleic acids and bound factors in this assay system.

1. A method for isolating or purifying a biomolecule directly or indirectly bound to a region of interest of a nucleic acid in a cell or a virus, comprising:
a) providing a cell or plurality of cells;
b) contacting or exposing the cell or cells or virus to a fixing or cross-linking agent or treatment under conditions in which direct or indirect binding of one or more biomolecules to the nucleic acid is preserved or maintained, thereby producing fixed or cross-linked nucleic acid;
c) optionally isolating the fixed or cross-linked nucleic acid;
d) fragmenting the fixed or cross-linked nucleic acid, thereby producing fragmented fixed or cross-linked nucleic acid;
e) optionally denaturing the fragmented fixed or cross-linked nucleic acid;
f) hybridizing a capture oligonucleotide to the fragmented fixed or cross-linked nucleic acid to form a hybridized target, wherein the capture oligonucleotide is complementary to all or a part of the region of interest of the nucleic acid; and
g) isolating or purifying the hybridized target, thereby isolating or purifying a biomolecule directly or indirectly bound to a region of interest of the nucleic acid in the cell or the virus.

2. A method for isolating or purifying a biomolecule that directly or indirectly binds to a region of interest of a nucleic acid in a cell or a virus, comprising:
a) providing a cell or plurality of cells or a virus;
b) contacting or exposing the cell or cells to a fixing or cross-linking agent or treatment under conditions in which direct or indirect binding of one or more biomolecules to the nucleic acid is preserved or maintained, thereby producing fixed or cross-linked nucleic acid;
c) optionally isolating the fixed or cross-linked nucleic acid;
d) fragmenting the fixed or cross-linked nucleic acid, thereby producing fragmented fixed or cross-linked nucleic acid;
e) optionally denaturing the fragmented fixed or cross-linked nucleic acid;
f) hybridizing a capture oligonucleotide to the fragmented fixed or cross-linked nucleic acid to form a hybridized target, wherein the capture oligonucleotide is complementary to all or a part of the region of interest of the nucleic acid;
g) isolating or purifying the hybridized target; and
h) removing one or more biomolecules directly or indirectly bound to the hybridized target, thereby isolating or purifying a biomolecule that directly or indirectly binds to the region of interest of the nucleic acid in the cell or the virus.

3. The method of claim 1, wherein the nucleic acid comprises eukaryotic, bacterial or viral DNA or RNA.

4.-6. (canceled)

7. The method of claim 1, wherein the nucleic acid comprises a tissue or organ sample, a biological fluid, a cell lysate, soil, water, air, or a synthetically produced biomolecule or mixture of biomolecules.

8. The method of claim 1, wherein the biomolecule is a polypeptide or nucleic acid.

9. The method of claim 1, wherein step b) comprises exposure to radiation.

10. The method of claim 9, wherein the radiation comprises ultraviolet (UV) radiation, laser radiation or light radiation.

11. The method of claim 1, wherein the fixing or cross-linking agent has at least two functional groups, or wherein treatment is with two or more fixing or cross-linking agents.

12. The method of claim 11, wherein the fixing or cross-linking agent or treatment is a chemical cross-linker.

13. The method of claim 12, wherein the chemical cross-linker is reversible.

14. The method of claim 1, wherein fixing or cross-linking agent or treatment is or has one or more of: an aldehyde-containing compound; an amine reactive group; a sulfhydryl reactive group; a hydroxyl reactive group; a carboxyl-reactive compound; a carbohydrate reactive group; an alkylating agent; a nucleic acid base reactive group; a nucleic acid backbone alkylating agent; or a nucleic acid adduct-forming metallic compound.

15. The method of claim 14, wherein the aldehyde-containing compound is Formaldehyde, glyoxal or glutaraldehyde.

16.-37. (canceled)

38. The method of claim 1, wherein the fixing or cross-linking agent comprises a compound having a nucleic acid reactive or binding group and a protein reactive or binding group.

39.-45. (canceled)

46. The method of claim 1, wherein the fixing or cross-linking agent or treatment comprises two or more fixing or cross-linking agents.

47. The method of claim 1, wherein the fixing or cross-linking agent or treatment comprises two or more cross linkers.

48.-57. (canceled)

58. The method of claim 1, wherein the capture oligonucleotide is labeled with an affinity tag or a radionuclide or immobilized on a substrate, or wherein the fixative or cross-linking agent or treatment is labeled with a radionuclide or electron spin-label.

59. (canceled)

60. The method of claim 58, wherein the substrate comprises a membrane, glass, plastic or other synthetic or natural polymer, metal or silica.

61. The method of claim 58, wherein the substrate comprises a plurality of capture oligonucleotides.

62. The method of claim 58, wherein the capture oligonucleotides are located at defined positions on the substrate.

63.-67. (canceled)

68. The method of claim 1, wherein step g) is performed by physically separating the hybridized target from non-hybridized fragmented fixed or cross-linked nucleic acid.

69. The method of claim 2, wherein step g) is performed by thermal, chemical, enzymatic or mechanical disruption of the biomolecule bound to the hybridized target, or any combination thereof.

70.-72. (canceled)

73. The method of claim 1, further comprising, step h) characterizing or identifying the isolated or purified biomolecule bound to the region of interest of the nucleic acid, or step i) characterizing or identifying one or more of the removed biomolecules.

74. The method of claim 73, wherein step h) or i) comprises gel electrophoresis, western blotting, mass spectrometry, matrix-assisted Laser Desorption Ionization (MALDI), chromatography, nuclear magnetic resonance, or crystallography.

75. The method of claim 1, further comprising, step h) characterizing or identifying the nucleic acid sequence or residues thereof to which one or more of the biomolecules bind.

76.-88. (canceled)

89. A method for identifying or screening a compound that modulates binding of a biomolecule directly or indirectly to a region of interest of a nucleic acid in a cell or virus, comprising:
a) providing a cell or plurality of cells or virus;
b) contacting the cell or cells or virus with a test compound;
c) contacting or exposing the cell or cells or virus to a fixing or cross-linking agent or treatment under conditions in which direct or indirect binding of one or more biomolecules to the nucleic acid is preserved or maintained, thereby producing fixed or cross-linked nucleic acid;
d) optionally isolating the fixed or cross-linked nucleic acid;
e) fragmenting the fixed or cross-linked nucleic acid, thereby producing fragmented fixed or cross-linked nucleic acid;
f) optionally denaturing the fragmented fixed or cross-linked nucleic acid;
g) hybridizing a capture oligonucleotide to the fragmented fixed or cross-linked nucleic acid to form a hybridized target, wherein the capture oligonucleotide is complementary to all or a part of the region of interest of the nucleic acid;
h) isolating or purifying the hybridized target;
i) identifying or characterizing one or more biomolecules directly or indirectly bound to the region of interest of the nucleic acid; and
j) comparing binding of the one or more biomolecules to binding in the absence of the test compound, wherein altered binding in the presence of the test compound identifies the compound as a compound that modulates binding of the biomolecule to the region of interest of the nucleic acid in the cell or virus.

90. The method of claim 89, wherein the test compound is a drug.
